(19) United States
(12) Patent Application Publication
(10) Pub. No.: US 2021/0154635 A1
(43) Pub. Date: May 27, 2021

(54) APPARATUSES FOR DEHYDROGENATION OF ALKANES

(71) Applicant: INDIAN OIL CORPORATION LIMITED, Mumbai (IN)

(72) Inventors: Hima Bindu DOOSA, Faridabad (IN); Ram Mohan THAKUR, Faridabad (IN); Vineeth Venu NATH, Faridabad (IN); Sadhullah MUKTHIYAR, Faridabad (IN); Madhusudan SAU, Faridabad (IN); Debasis BHATTACHARYYA, Faridabad (IN); Gurpreet Singh KAPUR, Faridabad (IN); Sankara Sri Venkata RAMAKUMAR, Faridabad (IN)

(73) Assignee: INDIAN OIL CORPORATION LIMITED, Mumbai (IN)

(21) Appl. No.: 17/035,461

(22) Filed: Sep. 28, 2020

(30) Foreign Application Priority Data
Nov. 27, 2019 (IN) 201921048665

(51) Int. Cl. B01J 8/18; C07C 5/333; B01J 8/00

(52) U.S. Cl. CPC B01J 8/1863; C07C 5/333; B01J 8/1872; B01J 8/0055; B01J 8/008; B01J 2208/00991; B01J 2208/0038; B01J 2208/0053; B01J 2208/00654; B01J 2208/00938; B01J 2208/00371

(57) ABSTRACT

The present disclosure relates to circulating fluidized bed apparatuses for dehydrogenation of alkanes to alkenes with higher yield and selectivity. The apparatus includes a riser-type reactor, a separator section, a regenerator and a withdrawal well disposed downstream to the regenerator. The apparatus includes a transfer line to receive hot regenerated catalyst free of oxygen from the withdrawal well, and to pre-treat the catalyst with a reducing gas to regulate-oxidation state of metals on the catalyst before reintroducing the catalyst to the riser-type reactor. The transfer line is formed in an elongated U-shaped pipe such that the oxidation state of the metals on the catalyst is regulated by the time the pre-treated catalyst reaches the bottom of the riser-type reactor.

### FIELD OF THE INVENTION

The present disclosure relates to dehydrogenation of alkanes and particularly, relates to apparatuses for dehydrogenation of alkanes to alkenes.

### BACKGROUND OF THE INVENTION

With the increasing global demand for petrochemicals, the production of petrochemical precursors, such as, ethylene, propylene, and butylenes has gained momentum. Catalytic dehydrogenation is one of the well-known processes to produce light alkenes from their respective alkanes. Catalytic dehydrogenation of alkanes is a fast and equilibrium limited reaction, wherein high temperatures and low pressures favor the formation of alkene product. During the process of alkane dehydrogenation, the catalyst gradually gets deactivated, primarily due to the formation of coke. The catalyst is then regenerated by combusting the deposited coke in the presence of air or oxygen periodically. Since the dehydrogenation reaction is endothermic, external heat is supplied either by pre-heating the feed to high temperatures or by employing heaters in between the reactors connected in series, in addition to the heat generated by the combustion of carbonaceous deposits on the catalyst.

Several types of reactor configurations or apparatus for catalytic dehydrogenation of alkanes were reported in literature. U.S. Pat. No. 5,436,383 discloses a catalytic dehydrogenation system wherein, a fixed bed, moving bed, or fluid bed reactor can be employed. In case of fixed bed reactor system, as described in the U.S. Pat. No. 6,392,113B1, a set of catalytic dehydrogenation reactors are operated in a cyclic non-steady-state mode with regeneration of a catalyst bed every 10 to 30 minutes. The catalyst bed is heated during regeneration and this heat is used to carry out the dehydrogenation reaction. Large sized multiple reactors connected in parallel are required for large plant sizes. Frequent cycling of the system can lead to operational and maintenance problems and the non-continuous system is thermally less efficient than a continuous process.

U.S. Pat. No. 5,130,106 discloses moving bed radial flow reactors connected in series with continuous catalyst regeneration system for catalytic alkane dehydrogenation process. However, intermediate heaters are required to meet the heat demand of the process. In order to avoid the large size reactors and inter-heaters, and thereby reduce the capital and operational cost, circulating fluidized bed reactor system with continuous catalyst regeneration appears to be a smart choice. The circulating fluidized bed reactor systems have several advantages, such as, easy catalyst addition and withdrawal, continuous regeneration of catalyst, changing one catalyst formulation to other without shutdown, flexibility to introduce any type of catalyst additive to alter the yield pattern, etc.

Typical catalysts used for the dehydrogenation of light alkanes are Pt—Sn, oxides of Cr, V, etc. After the alkane dehydrogenation reaction, the catalyst needs to be regenerated in the presence of oxygen containing gas to remove the coke deposits. However, during the regeneration process, the active metals on the catalyst get oxidized due to which the performance of the catalyst may alter. In order to maintain high product selectivity, the oxidation state of the metals on the catalyst needs to be regulated. The present invention discloses several reactor configurations to achieve high alkene yield and selectivity by addressing the above-mentioned issues.

### SUMMARY OF THE INVENTION

This summary is provided to introduce a selection of concepts, in a simplified format, that are further described in the detailed description of the invention. This summary is neither intended to identify key or essential inventive concepts of the invention and nor is it intended for determining the scope of the invention.

In an embodiment of the present disclosure, a circulating fluidized bed apparatus for dehydrogenation of alkanes is disclosed. The apparatus includes a riser-type reactor adapted to accommodate a pre-heated alkane feed stream and a catalyst for reaction, and a regenerator adapted to burn coke deposited on the catalyst and to heat the catalyst to desired temperature. The apparatus includes a withdrawal well disposed downstream to the regenerator and adapted to receive regenerated catalyst and to remove air from pores of the regenerated catalyst. The apparatus includes a transfer line connecting the withdrawal well with the riser-type reactor and adapted to receive hot regenerated catalyst free of oxygen from the withdrawal well, and to pre-treat the catalyst with a reducing gas to regulate oxidation state of metals on the catalyst before reintroducing the catalyst to the bottom of the riser-type reactor. The transfer line is formed in an elongated U-shaped pipe such that the oxidation state of the metals on the catalyst is regulated by the time the catalyst reaches the bottom of the riser-type reactor.

In another embodiment of the present disclosure, a circulating fluidized bed apparatus for dehydrogenation of alkanes is disclosed. The apparatus includes a riser-type reactor adapted to accommodate a pre-heated alkane feed stream and a catalyst for reaction, and a regenerator adapted to burn coke deposited on the catalyst and to heat the catalyst to desired temperature. The apparatus includes a withdrawal well disposed downstream to the regenerator and adapted to receive regenerated catalyst and to remove air from pores of the regenerated catalyst. The apparatus includes a vessel connected to the withdrawal well and adapted to receive hot regenerated catalyst free of oxygen from the withdrawal well, and to pre-treat the catalyst with a reducing gas to regulate oxidation state of metals on the catalyst before reintroducing the catalyst to a second end of the riser-type reactor. The second end is submerged in the fluidized bed of catalyst in the vessel.

In another embodiment of the present disclosure, a circulating fluidized bed apparatus for dehydrogenation of alkanes is disclosed. The apparatus includes a riser-type reactor adapted to accommodate a pre-heated alkane feed stream and a catalyst for reaction, and a separator coupled to the riser-type reactor. The apparatus includes a regenerator disposed downstream to the separator and adapted to receive a fraction of hydrocarbon-free catalyst after the reaction and stripping, facilitate regeneration of spent catalyst by burning of coke deposited on the catalyst, and heat the catalyst to a desired temperature. The apparatus includes a holding vessel disposed downstream to the separator and adapted to receive the remaining fraction of hydrocarbon-free catalyst. The regenerator and the holding vessel are coupled to the bottom of the riser-type reactor through a third standpipe and a fourth standpipe, respectively, delivering the respective fraction of the catalyst post treatment to the bottom of the riser-type reactor.

To further clarify the advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates a schematic view of an apparatus for dehydrogenation of alkanes, according to an embodiment of the present disclosure;

FIG. 2 illustrates a schematic view of an apparatus for dehydrogenation of alkanes, according to another embodiment of the present disclosure; and

FIG. 3 illustrates a schematic view of an apparatus for dehydrogenation of alkanes, according to another embodiment of the present disclosure.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

### DETAILED DESCRIPTION OF THE DRAWINGS

For promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates. Unless otherwise defined, all technical and scientific terms used herein have the same meaning as commonly understood by one of ordinary skilled in the art to which this invention belongs. The system, methods, and examples provided herein are illustrative only and not intended to be limiting.

Embodiments of the present invention will be described below in detail with reference to the accompanying drawings.

The present disclosure relates to catalytic dehydrogenation of alkanes to alkenes and particularly, relates to apparatuses for the dehydrogenation of light alkanes (C₂-C₅) to alkenes and more particularly, to a circulating fluidized bed apparatus for the dehydrogenation of light alkanes to alkenes.

One of the objectives of the present invention is to provide an apparatus to produce high-value olefins, such as propylene and iso-butylene. Another objective is to provide a process which enhances the olefin selectivity by providing efficient contact between catalyst and alkane feed and by regulating the oxidation state of active metals on the catalyst. Another objective is to improve the catalyst life by regeneration of fraction of the catalyst.

The present invention provides an apparatus to produce light olefins at higher selectivities by catalytic dehydrogenation of corresponding paraffins. The apparatus comprises of circulating riser reactor with regeneration of partial or complete catalyst. The higher yield and selectivity of alkenes are achieved by providing efficient contact between the feed and the circulating catalyst and by regulating the oxidation states of the metals on catalyst. The disclosed reactor configurations allow flexibility during operation in terms of catalyst/additive addition/withdrawal, etc., and do not require any inter-heaters or large size reactors.

Several reactor configurations for catalytic dehydrogenation of alkanes to alkenes, comprising of circulating fluidized bed reactor system with continuous catalyst regeneration are described. The catalyst and the feed flow co-currently in the riser type of reactor, wherein the dehydrogenation reaction occurs, followed by stripping of product gases from the catalyst in the stripper. A part of the catalyst or complete catalyst is regenerated, using air or oxygen or mixture of air and fuel, depending upon the amount of coke formed and quantity of heat required for the dehydrogenation reaction. In case of complete regeneration of the catalyst, the regenerated catalyst is treated with a reducing agent prior to the reaction either in an elongated standpipe pipe or in a vessel. The process of the present invention is exemplified by, but not limited to the following figures.

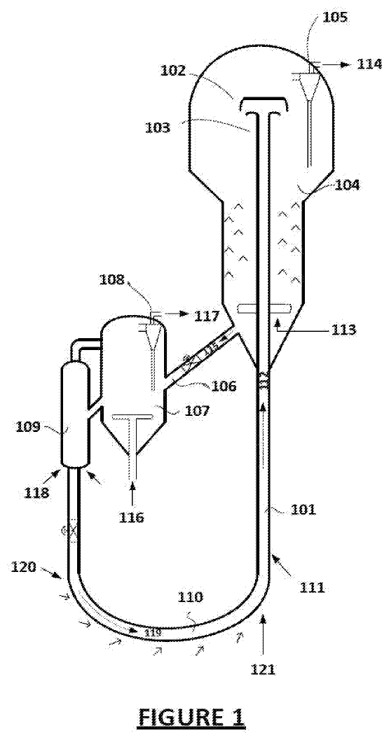

FIG. 1 illustrates a schematic view of an apparatus 100 for dehydrogenation of alkanes, according to an embodiment of the present disclosure. The apparatus 100 may also be understood as a circulating fluidized bed reactor system with complete catalyst regeneration.

The apparatus 100 may include, but is not limited to, a riser-type reactor 101, a separator 104 coupled to the riser-type reactor 101, a regenerator 107 coupled to the separator 104, a withdrawal well 109 disposed downstream to the regenerator 107, and a transfer line 110 connecting the withdrawal well 109 with the riser-type reactor 101. The separator 104 includes a riser termination device 102, a set of cyclones, and a stripper. In an embodiment, a first end 103 of the riser-type reactor 101 may be opening into the separator 104. In an embodiment, the apparatus 100 may include the riser termination device 102 formed at the first end 103 of the riser-type reactor 101 opening into the stripper through the set of cyclones.

The riser-type reactor 101 may be adapted to accommodate a pre-heated alkane feed stream 111 and a catalyst 112 for reaction. The reaction may occur in the riser-type reactor 101. The pre-heated alkane feed stream 111 with or without diluents is contacted with co-currently upward moving catalyst 112 in the riser-type reactor 101. In an embodiment, the alkanes include, but are not limited to, ethane, propane, n-butane, iso-butane, and any combination thereof. The diluent includes, but is not limited to, steam, nitrogen, and any other inert gas. The alkanes are dehydrogenated to their respective alkenes in the riser-type reactor 101.

In an embodiment, after the reaction, the catalyst 112, an alkene product, unreacted alkanes and other gases may be introduced into the separator 104, wherein the alkene product, unreacted alkanes and other gases are separated from the catalyst 112 in the riser termination device 102 and the set of cyclones. The gases including hydrocarbons 114 are expelled through a vent 105 for further separation or purification, and the catalyst falls into the stripper section. The stripper may be adapted to receive the catalyst, the remaining entrapped hydrocarbons, and gases from the cyclones. Further, the stripper may facilitate removal of remaining hydrocarbons and gases from the catalyst for generation of hydrocarbon vapours. In an embodiment, the hydrocarbons are stripped off from the catalyst using steam or nitrogen or any other inert gas 113 or any suitable gaseous stream. The stripper packing and internals are of any suitable design. The hydrocarbon vapours may include, but are not limited to, the alkene product and the unreacted alkanes. The separator 104 may be coupled to the regenerator 107.

In an embodiment, the apparatus 100 may include a standpipe 106 adapted to connect the separator 104 with the regenerator 107. Further, the apparatus 100 may include a slide valve disposed in the standpipe 106. The slide valve may be adapted to regulate a flow of the hydrocarbon-free catalyst 115 from the separator 104 to the regenerator 107.

The regenerator 107 may be adapted to receive the hydrocarbon-free catalyst, facilitate burning of coke deposited on the catalyst, heat the catalyst to a desired temperature, and separate catalyst fines from a flue gas generated. In an embodiment, the desired temperature may vary within a range of 600 to 800 Degrees Celsius. In an embodiment, air or oxygen or mixture of air and fuel 116 are supplied to burn the coke deposited on the catalyst and to heat the catalyst to the desired temperature. The air distributor in the regenerator is of any standard design. In an embodiment, the regenerator 107 may include a vent 108 adapted to expel the flue gas. For example, the catalyst fines are separated from the flue gas 117 using a set of cyclones and the flue gas is vented out from the regenerator 107. The regenerator 107 may be coupled with the withdrawal well 109.

The withdrawal well 109 may be adapted to receive the regenerated catalyst from the regenerator 107 and remove air from the pores of the regenerated catalyst. In an embodiment, the air from catalyst pores is stripped off using steam or nitrogen 118. Further, the withdrawal well 109 may be connected with the riser-type reactor 101 through the transfer line 110. In an embodiment, the apparatus 100 may also include a slide valve disposed in the transfer line 110 to regulate the catalyst flow. The transfer line 110 may be adapted to receive hot regenerated catalyst free of oxygen from the withdrawal well 109. Subsequently, the transfer line 110 may regulate an oxidation state of metals on the catalyst by treating with a reducing gas 120 before reintroducing the catalyst to the bottom of the riser-type reactor 101. In an embodiment, the reducing gas 120 may include, but is not limited to, hydrogen, methane, fuel gas, and dry gas. In an embodiment, the transfer line 110 may be in form of an elongated U-shaped pipe. The construction of the transfer line 110 is such that the oxidation state of the metals on the catalyst is regulated by the time the pre-treated catalyst reaches the bottom of the riser-type reactor 101. In case of the proposed U-shaped pipe, the reducing gas has sufficient time to regulate the oxidation state of the metals on the catalyst. The pre-treated catalyst 112 is then lifted in the riser-type reactor 101 by using steam or nitrogen 121 for the dehydrogenation reaction.

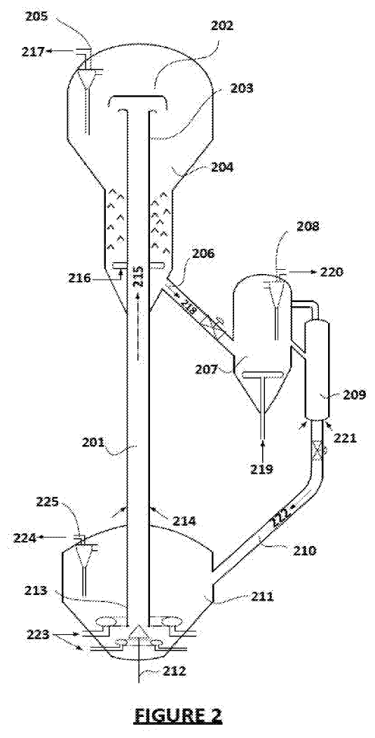

FIG. 2 illustrates a circulating fluidized bed apparatus 200 for dehydrogenation of the alkanes, according to another embodiment of the present disclosure. The apparatus 200 is suitable for catalysts containing active metals/components of lower oxidizing strength.

The apparatus 200 may include, but is not limited to, the riser-type reactor 201, the separator 204 coupled to the riser-type reactor 201, the standpipe 206 connecting the separator 204 and the regenerator 207, the slide valve disposed in the standpipe 206, the regenerator 206 having the vent 208, the withdrawal well 209 disposed downstream to the regenerator 207, the transfer line 210 connecting the withdrawal well 209 with a vessel 211. For the sake of brevity, constructional and operational features of the components that are already explained in the description of FIG. 1 are not explained in detail in the description of FIG. 2. For example, the riser-type reactor 201, the separator 204 comprising of the riser termination device 202, cyclones, stripper, and the vent 205, the standpipe 206, the regenerator 207, the regenerator vent 208, the withdrawal well 209, and the transfer line 210 operate in the similar manner as explained in FIG. 1. Therefore, treatment of the catalyst, the alkanes, and the alkene product is about same in the apparatus 200 as well.

The vessel 211 is a component which was not disclosed in the apparatus 100. The vessel 211 may be connected to the withdrawal well 209 through the transfer line 210. As illustrated, the construction of the transfer line 210 in the apparatus 200 is different from the apparatus 100, for example, in order to facilitate connection of the withdrawal well 209 with the vessel 211. The vessel 211 may be adapted to receive hot regenerated catalyst free of oxygen from the withdrawal well 209. Further, the vessel 211 may be adapted to pre-treat the catalyst with a reducing gas to regulate oxidation state of metals on the catalyst before reintroducing the catalyst to a second end 213 of the riser-type reactor 201. The second end 213 may be submerged in a fluidized bed of catalyst in the vessel 211. The vessel 211 may comprise a vent 225.

Therefore, in the vessel 211, the catalyst is subjected to reduction by using the reducing agent 223, such as, hydrogen and methane, and the flue gas 224 generated is sent out through the cyclones. In an example, the residence time of the catalyst in the vessel 211 may be about 2 to 6 minutes. The pre-treated catalyst 215 from the bed moves upward into the riser-type reactor 102 for the dehydrogenation of alkanes to alkenes.

In an embodiment, the apparatus 200 may include a plug valve 212 disposed at the bottom of the vessel 211 and adapted to regulate a flow of the catalyst into the rise-type reactor 201. In an embodiment, the plug valve 212 may be disposed at the center of the bottom of the vessel 211.

In an embodiment, the apparatus 200 may include at least one gas distributor of suitable size and design located at varying height at the bottom of the vessel 211. Further, the apparatus 200 may include at least one feed injector disposed at just above the vessel 211.

The apparatus 200 is suitable for catalysts containing active metals/components of lower oxidizing strength.

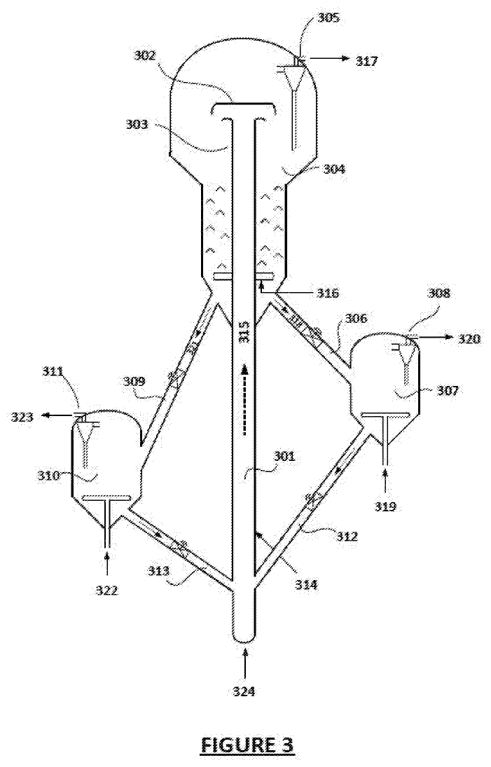

FIG. 3 illustrates a circulating fluidized bed apparatus 300 for dehydrogenation of the alkanes to the alkenes, according to an embodiment of the present disclosure. For the sake of brevity, constructional and operational features of present disclosure that are already explained in the description of FIG. 1 and FIG. 2 are not explained in detail in the description of FIG. 3.

The apparatus 300 may include, but is not limited to, the riser-type reactor 301 adapted to accommodate the pre-heated alkane feed stream 314 and the catalyst 315 for the reaction, and the separator 304 coupled to the riser-type reactor 301. The separator 304 may be adapted to receive the catalyst, the alkene product, and the unreacted alkanes, and facilitate removal of the hydrocarbons from the catalyst for generation of the hydrocarbon vapours.

The apparatus 300 may further include the regenerator 307 disposed downstream to the separator 304 through a first standpipe 306. The regenerator 307 may be adapted to receive a fraction of the hydrocarbon-free catalyst, facilitate burning of coke deposited on the catalyst, heat the catalyst to a desired temperature, and separate catalyst fines from the flue gas generated.

The apparatus 300 may further include a holding vessel 310 disposed downstream to the separator 304 through a second standpipe 309. The holding vessel 310 may be adapted to receive the remaining fraction of hydrocarbon-free catalyst and heat the catalyst to a desired temperature. The regenerator 307 and the holding vessel 310 may be coupled to the bottom of the riser-type reactor through a third standpipe 312 and a fourth standpipe 313, respectively, delivering the respective fraction of the catalyst post treatment to the bottom of the riser-type reactor 301. In an embodiment, a feed inlet in the riser-type reactor 301 may be located at an altitude where the uniform mixing of the spent and regenerated catalyst or uniform temperature distribution is achieved.

In an embodiment, altitudes of the regenerator 307 and the holding vessel 310 may be selected based on an overall pressure balance depending on the respective fraction of the catalyst delivered to the regenerator 307 and the holding vessel 310.

Since the contact time of alkane feed and the catalyst in the riser-type reactors is very short, typically, in the range of 0.1-5.0 seconds, product selectivity would be higher and the amount of coke formed during the dehydrogenation is lower when compared to that in fixed bed or moving bed reactors. Therefore, it is not necessary to regenerate the complete catalyst in every cycle. Thus, a fraction of the spent catalyst 318 is sent to the regenerator 307, wherein the coke deposited on the catalyst is combusted and the catalyst is heated to the desired temperature in the presence of air or oxygen or air and fuel gas mixture 319. The flue gas 320 generated exits the regenerator 307 through cyclones and the hot regenerated catalyst fraction flows down through the third standpipe 312 to the bottom of the riser-type reactor 301.

The remaining fraction of the catalyst 321 flows through the second standpipe 309 to the catalyst holding vessel 310, wherein the catalyst is heated using a hot inert gas 322. The relatively cold inert gas 323 exits the catalyst holding vessel 310 from the cyclones and the hot spent catalyst fraction enters the bottom of the riser-type reactor 301 through the fourth standpipe 313. Both the catalyst fractions get mixed at the riser bottom and get lifted along the riser 301 using steam or nitrogen 324. Since only a part of the catalyst is subjected to regeneration, the overall life of catalyst is improved along with regulation the oxidation states of the metals in the catalyst.

As would be appreciated by a person skilled in the art, the treatment of the catalyst and the other substances through the apparatus 200 and the apparatus 300 is similar to the treatment as explained for the apparatus 100. For the sake of brevity, such details are not repeated.

As would be gathered, the apparatuses 100, 200, and 300 are adapted to dehydrogenate the alkanes to alkenes, particularly, light alkanes with carbon number ranging from 2 to 5, wherein the dehydrogenation reaction occurs in the circulating riser reactor with regeneration of partial or complete catalyst. The alkene product yield and selectivity are enhanced by providing efficient contact between the feed and the circulating catalyst and by regulating the oxidation state of the active metals on the catalyst. Some of the advantages of the apparatuses 100, 200, and 300 of the present disclosure are:
Production of high-value olefins, particularly, propylene and iso-butylene from their respective alkanes, with higher selectivity.
Ease & flexibility in operation.
Enhancement of catalyst life.
No requirement of inter-heaters or large size reactors.
Continuous catalyst addition and withdrawal without unit shutdown.
Reaction and regeneration occur in separate sections and thus, no intermixing of hydrocarbons with oxygen/air.
Disclosed apparatus typically operates at pressures above atmospheric pressures, and hence, no possibility of permeation of ambient air into the system.

The U-shaped profile of the transfer line 110 of the apparatus 100 ensures that the oxidation state of the metals on the regenerated catalyst is regulated by the time the catalyst reaches the bottom of the riser-type reactor 102. Further, the apparatus 200 is suitable for catalysts containing active metals/components of lower oxidizing strength. Moreover, the catalyst life can be enhanced by apparatus 300 by not subjecting the entire catalyst for regeneration. Desired olefin selectivity is achieved by such proportionate mixing of the regenerated catalyst and spent catalyst in apparatus 300 due the regulation of oxidation states of the overall equilibrium catalyst.

Therefore, the apparatuses 100, 200, and 300 are constructed to offer a comprehensive approach for dehydrogenation of the alkanes to alkenes in different scenarios. As would be gathered from above, the apparatuses 100, 200, and 300 are simple, effective, easy, and flexible to operate, and cost-effective.

While specific language has been used to describe the present disclosure, any limitations arising on account thereto, are not intended. As would be apparent to a person in the art, various working modifications may be made to the method in order to implement the inventive concept as taught herein. The drawings and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment.

1. A circulating fluidized bed apparatus for dehydrogenation of alkanes comprising a riser-type reactor, a separator coupled to the riser-type reactor, a regenerator coupled to the separator and a withdrawal well disposed downstream to the regenerator, and a transfer line connecting the withdrawal well with the riser-type reactor, the transfer line adapted to:
receive hot regenerated catalyst free of oxygen from the withdrawal well; and
pre-treat the catalyst with a reducing gas to regulate the oxidation state of metals on the catalyst before reintroducing the catalyst to the bottom of the riser-type reactor, wherein the transfer line is formed in an elongated U-shaped pipe such that the oxidation state of the metals on the catalyst is regulated by the time the catalyst reaches the bottom of the riser-type reactor.

2. The circulating fluidized bed apparatus as claimed in claim 1, wherein the riser-type reactor is adapted to accommodate a pre-heated alkane feed stream and a catalyst for dehydrogenation reaction.

3. The circulating fluidized bed apparatus as claimed in claim 1, wherein the separator comprising:
a riser termination device for disengaging the catalyst and hydrocarbons;
a set of cyclones for separation of catalyst and hydrocarbon vapours; and
a stripper for stripping out entrapped hydrocarbons from the catalyst by using a stripping media including one of steam, nitrogen, or any suitable gaseous stream, wherein the hydrocarbons comprising the alkene product and the unreacted alkanes.

4. The circulating fluidized bed apparatus as claimed in claim 1, wherein the regenerator is adapted to:
receive the spent catalyst from the separator after hydrocarbon removal through a standpipe at a rate controlled by a slide valve in the standpipe; and
facilitate regeneration of the catalyst by burning the coke deposited on the catalyst and heating the catalyst to a desired temperature.

5. The circulating fluidized bed apparatus as claimed in claim 1, wherein the withdrawal well is adapted to receive regenerated hot catalyst and to remove air from the pores of the regenerated catalyst.

6. The circulating fluidized bed apparatus as claimed in claim 1, wherein the reducing gas comprising at least one of hydrogen, methane, fuel gas and dry gas.

7. A circulating fluidized bed apparatus for dehydrogenation of alkanes comprising a riser-type reactor, a separator coupled to the riser-type reactor, a regenerator coupled to the separator, and a withdrawal well disposed downstream to the regenerator and a vessel connected to the withdrawal well through a transfer line, the vessel adapted to:
receive hot regenerated catalyst free of oxygen from the withdrawal well; and
pre-treat the catalyst with a reducing gas to regulate the oxidation state of metals on the catalyst before reintroducing the catalyst to a second end of the riser-type reactor, wherein the second end is submerged in a fluidized bed of catalyst in the vessel.

8. The circulating fluidized bed apparatus as claimed in claim 7, wherein the riser-type reactor is adapted to accommodate a pre-heated alkane feed stream and a catalyst for dehydrogenation reaction.

9. The circulating fluidized bed apparatus as claimed in claim 7, wherein the separator comprising:
a riser termination device for disengaging the catalyst and hydrocarbons;
a set of cyclones for separation of catalyst and hydrocarbon vapours; and
a stripper for stripping out entrapped hydrocarbons from the catalyst by using a stripping media including one of steam, nitrogen, or any gaseous stream, wherein the hydrocarbon comprising the alkene product and the unreacted alkanes.

10. The circulating fluidized bed apparatus as claimed in claim 7, wherein the regenerator is adapted to:
receive the spent catalyst from the separator after hydrocarbon removal through a first standpipe at a rate controlled by a slide valve in the standpipe; and
facilitate regeneration of catalyst by burning the coke deposited on the catalyst and heating the catalyst to a desired temperature.

11. The circulating fluidized bed apparatus as claimed in claim 7, wherein the withdrawal well is adapted to receive regenerated catalyst from the regenerator and to remove air from the pores of the regenerated catalyst.

12. The circulating fluidized bed apparatus as claimed in claim 7, comprising a plug valve disposed at the bottom of the vessel and adapted to regulate a flow of the catalyst into the riser-type reactor.

13. The circulating fluidized bed apparatus as claimed in claim 7 comprising at least one gas distributor of suitable size and design located at varying height at the bottom of the vessel.

14. The circulating fluidized bed apparatus as claimed in claim 7 comprising at least one feed injector disposed at just above the vessel.

15. The circulating fluidized bed apparatus as claimed in claim 7, wherein the reducing gas comprising at least one of hydrogen or methane or fuel gas or dry gas.

16. A circulating fluidized bed apparatus for dehydrogenation of alkanes comprising:
a riser-type reactor adapted to accommodate a pre-heated alkane feed stream and a catalyst for dehydrogenation reaction;
a separator coupled to the riser-type reactor;
a regenerator disposed downstream to the separator and adapted to:
receive a fraction of hydrocarbon-free catalyst after the reaction and stripping;
facilitate regeneration of spent catalyst by burning of coke deposited on the catalyst; and
heat the catalyst to a desired temperature; and
a holding vessel disposed downstream to the separator and adapted to receive the remaining fraction of hydrocarbon-free catalyst,
wherein the regenerator and the holding vessel are coupled to the bottom of the riser-type reactor through a third standpipe and a fourth standpipe, respectively, delivering the respective fraction of the catalyst to the bottom of the riser-type reactor.

17. The circulating fluidized bed apparatus as claimed in claim 16, wherein the separator comprising:
a riser termination device for disengaging the catalyst and hydrocarbons;
a set of cyclones for separation of catalyst and hydrocarbon vapours; and
a stripper for stripping out entrapped hydrocarbons from the catalyst by using a stripping media including one of steam, nitrogen, and any gaseous stream, wherein the hydrocarbon comprising the alkene product and the unreacted alkanes.

18. The circulating fluidized bed apparatus as claimed in claim 16, wherein the holding vessel is adapted to:
receive remaining fraction of the hydrocarbon free stripped spent catalyst from the separator through the second standpipe at a rate controlled by slide valve in the second standpipe; and
provide hot oxygen free gas stream through the bottom to keep the catalyst under fluidized condition.

19. The circulating fluidized bed apparatus as claimed in claim 16, wherein altitudes of the regenerator and the holding vessel are selected based on an overall pressure balance depending on the respective fraction of the catalyst delivered to the regenerator and the holding vessel.
